(19) United States
(12) Patent Application Publication
(10) Pub. No.: US 2016/0193477 A1
(43) Pub. Date: Jul. 7, 2016

(54) System and Method for Treating a Vein

(71) Applicant: Cyanomed, Inc., Fallbrook, CA (US)

(72) Inventors: Scott Cote, Fallbrook, CA (US); Anthony Jakubowski, Montgomery, IL (US)

(73) Assignee: Cyanomed, Inc., Fallbrook, CA (US)

(21) Appl. No.: 14/972,436

(22) Filed: Dec. 17, 2015

Related U.S. Application Data
(60) Provisional application No. 62/099,166, filed on Jan. 1, 2015.

(51) Int. Cl. A61N 5/06; A61M 5/46; A61M 5/14

(52) U.S. Cl. CPC A61N 5/062; A61M 5/14; A61M 5/46; A61N 2005/0662; A61N 2005/0651; A61N 2005/0602

(57) ABSTRACT

A system and a method for treating a vein. The system is a curable composition with a mixture of N-butal Cyanoacrylate and/or 2-Octyl Cyanoacrylate with a photo initiator and the method provides steps of introducing the mixture into a patient to decrease the patient's procedure time and increase the bond strength between a vein's endothelial linings.

### RELATED APPLICATIONS

The present invention claims priority to co-pending U.S. Provisional Patent Application Ser. No. 62/099,166, filed 1 Jan. 2015.

### BACKGROUND OF THE INVENTION

Healthy leg veins contain valves that allow blood to move in one direction from the lower limbs toward the heart. These valves open when blood is flowing toward the heart, and close to prevent venous reflux, or the backward flow of blood. When veins weaken and become enlarged, their valves cannot close properly, which leads to venous reflux and impaired drainage of venous blood from the legs. Venous reflux is most common in the superficial veins. The largest superficial vein is the great saphenous vein, which runs from the top of the foot to the groin, where it originates at a deep vein.

Venous reflux can be classified as either asymptomatic or symptomatic, depending on the degree of severity. Symptomatic venous reflux disease is a more advanced stage of the disease and can have a profound impact on the patient's quality of life. People with symptomatic venous reflux disease may seek treatment due to a combination of symptoms and signs, which may include leg pain and swelling; painful varicose veins; skin changes such as discoloration or inflammation; and open skin ulcers.

A primary goal of treating symptomatic venous reflux is to eliminate the reflux at its source, such as, for example, the great saphenous vein. If a diseased vein is either closed or removed, blood can automatically reroute into other veins without any known negative consequences to the patient.

Non-invasive methods for treatment of venous reflux in the great saphenous vein include radiofrequency (RF) ablation, laser endothermal ablation, and sclerotherapy, including foam sclerotherapy. Radiofrequency ablation and laser ablation require tumescent anesthesia which produce both bruising and pain along the inner thigh and upper inner calf for several weeks, and both can have, side effects of burns and nerve damage. Radiofrequency ablation and laser ablation also require capital purchases of a radio frequency device or laser box in addition to expensive disposal mechanisms. While foam sclerotherapy is relatively non-invasive, it has a high rate of recurrence and potential side effects.

Another method for treatment comprises introducing a type of glue to the vein and inducing an external force to occlude the vein. The process displaces the blood and collapses the vein through the application of an external pressure force. While this procedure may reduce side effects and rate of recurrence, the glue may cure prematurely during the procedure and adhere the applicator to the inner wall of the vein which may cause damage to the vein when the applicator is removed, thus increasing the potential for complications and increasing patient recovery time.

### SUMMARY OF THE INVENTION

The present invention is directed towards systems for treating a vein. The system includes a kit that allows for delivery of a catheter into a vein to treat the vein. The catheter will allow delivery of a curable composition into the vein. The curable composition that is delivered into the vein is preferably a photo curable composition.

The present invention is also directed. towards a system for treating vein wherein the curable composition comprises a mixture of a photo initiator and a second compound. The second compound is preferably a cyanoacrylate composition.

The systems and kits of the present invention can also include a light source. Preferably, the light source is a light emitting diode (LED).

The present invention is also directed towards methods for treating a vein. The methods include delivering a composition into vein at a first position within the vein. The composition will be delivered by the use of a catheter. Once a predetermined treatment area is located within the vein, a bolus of the composition will be delivered from the catheter into the treatment area. The catheter may be retracted. The composition, which is a curable material, and preferably a photocurable material, will then be cured by use of a light source. The light source is also preferably a LED light source.

The methods of the present invention also include a further step of delivering a second bolus of the composition at a second treatment area within a vein. After a first bolus of material is delivered, the catheter can be further retracted in the vein, and the second bolus of the composition can be delivered. The second bolus can also be occluded with a light source, and preferably with an LED light source.

The present invention also includes a method of introducing a composition into a patient's vein and occluding the vein by curing the mixture with an LED light source.

The present invention also includes a method of using ultrasonic energy to occlude or cure the delivered composition.

The present invention also include methods of introducing a composition into a patient's vein that delivers boluses of the composition at more than one treatment area, with both ultrasonic energy and light energy to be used to occlude the composition.

The procedures and methods of the present invention reduces the potential for bonding of an applicator to the inner wall of the vein and also provides an increase in bond strength between the vein's endothelial linings.

### BRIEF DESCRIPTION OF THE DRAWINGS

FIG. 1 is a perspective view of a vein treatment kit according to the present invention.

FIG. 2 depicts an initial step in treating a vein according to the present invention, wherein a guide wire is inserted into a Great Saphenous Vein (GSV).

FIG. 3 demonstrates a further step in treating a vein according to the present invention, wherein an introducer sheath is inserted over the guide wire shown in FIG. 2.

FIG. 4 demonstrates a syringe being filled with a composition for use in the methods of the application.

FIG. 5 depicts the syringe of FIG. 4 being attached to a catheter.

FIG. 6 demonstrates the catheter of FIG. 5 being advance into the GSV.

FIG. 7 provide a further step of introducing the composition shown in FIG. 4 into the GSV.

FIG. 8 is a enlarged partially cut-away view of the GSV shown in FIG. 7, showing an occlusion of the composition being delivered in the vein.

FIG. 9 is a further step in treating a vein according to the present invention, wherein the composition delivered in the GSV is being cured with a light source.

FIG. 10 demonstrates a further step in treating a vein according to the present invention, wherein a second occlusion of the composition is being cured at a second position within the GSV.

FIG. 11 is a cut-away view of a treated vein according to the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

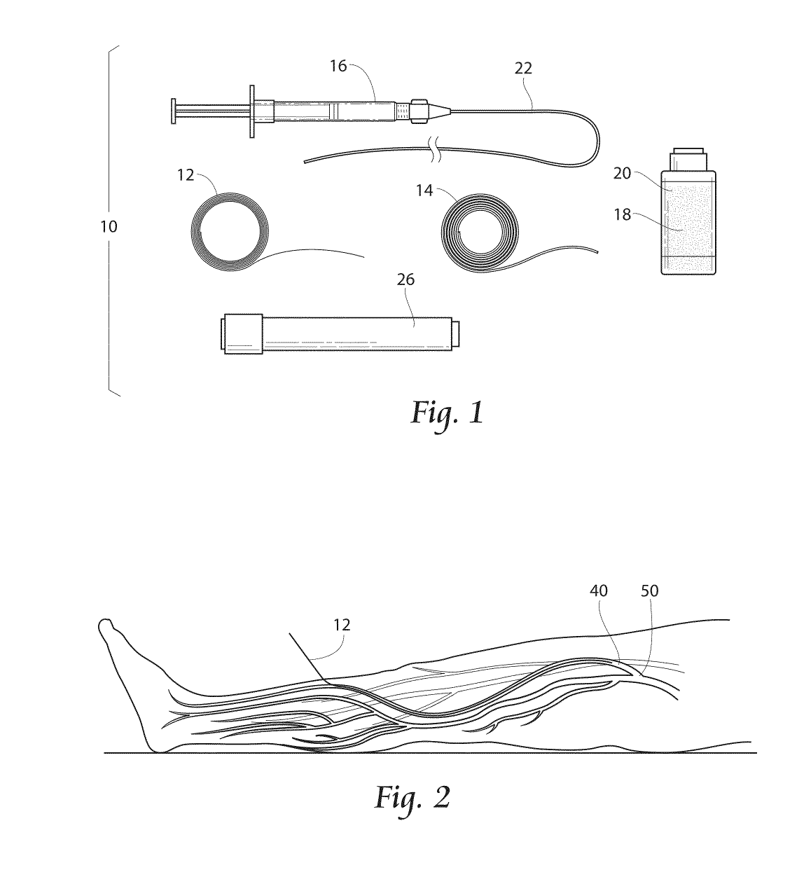

FIG. 1 illustrates a kit 10 for treating a vein 40 (FIG. 2) according to the present invention. As will be discussed further below, the kit 10 allows for the delivery of an occludable material into a vein 10 that reduces the potential for bonding of an applicator to the inner wall of the vein and also provides an increase in bond strength between the vein's endothelial linings 42 (FIG. 8) when compared to the prior art. The kit 10 includes a guide wire 12, which will provide access to an eventual treatment area or areas within a vein 40. The kit also includes an introducer sheath 14 which will assist in inserting a catheter 22 into a vein 40. As will be discussed in more detail below, the catheter 22 allows for the delivery of a mixture or composition 18 into a vein 40. The catheter 22 includes a distal insertion end 24 that will eventually be positioned at a predetermined treatment area or areas for delivery of the composition 18.

Still referring to FIG. 1, the kit 10 of the present invention also includes a syringe 16, which can be connected to the catheter 22. The syringe 16 is also designed so that it can be connected to a vial 20 that includes the composition or mixture 18. The composition 18 will be discussed in further detail below.

Referring to further to FIG. 1, the guide wire 12 is sized and configured so that it can be sufficiently inserted the length of a vein 40 (FIG. 2) for proper delivery of the composition. As an example and not as a limitation, the guide wire 12 may be a 150 cm long, 0.035 inch diameter guide wire, but it is understood that the size and dimensions of the guide wire 12 can be adjusted as necessary for proper introduction into a vein. It is also contemplated that a smaller guide wire (not shown), preferably having a diameter in the range of about 0.018 inch, may be advanced in the vein, followed by a micropuncture sheath/dilator (not shown), preferably having a gauge of about 5 Fr, and removing the smaller guide wire and micropuncture sheath/dilator prior to introducing and advancing the guide wire 12.

As noted above, the introducer sheath 14 assists in inserting the catheter 22 into the vein 40 (FIG. 2). The catheter 22 is sized and configured to be inserted inside of the introducer sheath 14, and the dimensions of each are designed for such an arrangement. As one example, the introducer sheath 14 is a 7 Fr, 24 cm introducer sheath, and the catheter 22 is a 5 Fr 80 cm HDPE 40-1110-01 catheter; however, it is contemplated that various forms of these elements may be used as is known in the art to provide safe navigation of the vein 40 (FIG. 2), arteries, lymphatics, etcetera.

The kit 10 shown in FIG. 1 also includes a light source 26. As will be discussed in further detail, below, the light source 26 provides energy that will allow the composition 18 to be cured. The light source 26 is preferably a light emitting diode (LED) light source. The emitted wavelength preferably may be considered as being an ultraviolet or infrared wavelength, with an ultraviolet light being preferred. As an example, the wavelength would be approximately 400-500 nm. In one embodiment, LED light source 26 preferably provides light having a wavelength of approximately 408 nm.

The kit 10 shown in FIG. 1 forms the basis for the methods of the present invention, which will be described in more detail in the Figures below. The methods described herein can be used for the treatment of a wide range of anatomical passageways, such veins, arteries, or lymphatics. The methods can also be used for both natural and artificial anatomical passageways. Similarly, a variety of conditions can be treated with the disclosed kit, system, and methods of the present invention. For example, the present inventions can be use to treat venous insufficiency/varicose veins of the upper and/or lower extremities, esophageal varices, gastric varices, hemorrhoidal varices, venous lakes, Klippel-Trenanay syndrome, telangiectasias, aneurysms, arterio-venous malformations, embolization of tumors or bleeding vessels, lymphedema, vascular and non-vascular fistulas, closure of fallopian tubes for sterilization, and other anomalies of the anatomical passageways.

As a way of example and not as a limitation, a preferred method for treating a vein, here a Great Saphenous Vein (GT) 40, is shown in FIGS. 2-10. FIG. 2 depicts the guide wire 12 being introduced into a patient's GSV 40. Prior to insertion, the patient will be prepped according to normal operating procedures. For example, the GSV 40 may be mapped, e.g. using ultrasonographic vein mapping or contrast venography, to visualize the patient's particular vascular anatomy. The entry site where the guide wire 12 is inserted will be sterilized and an anesthesia, e.g. lidocaine, can be administered. Once properly prepped, an incision will be made so that the guide wire 12 can be inserted into the GSV 40.

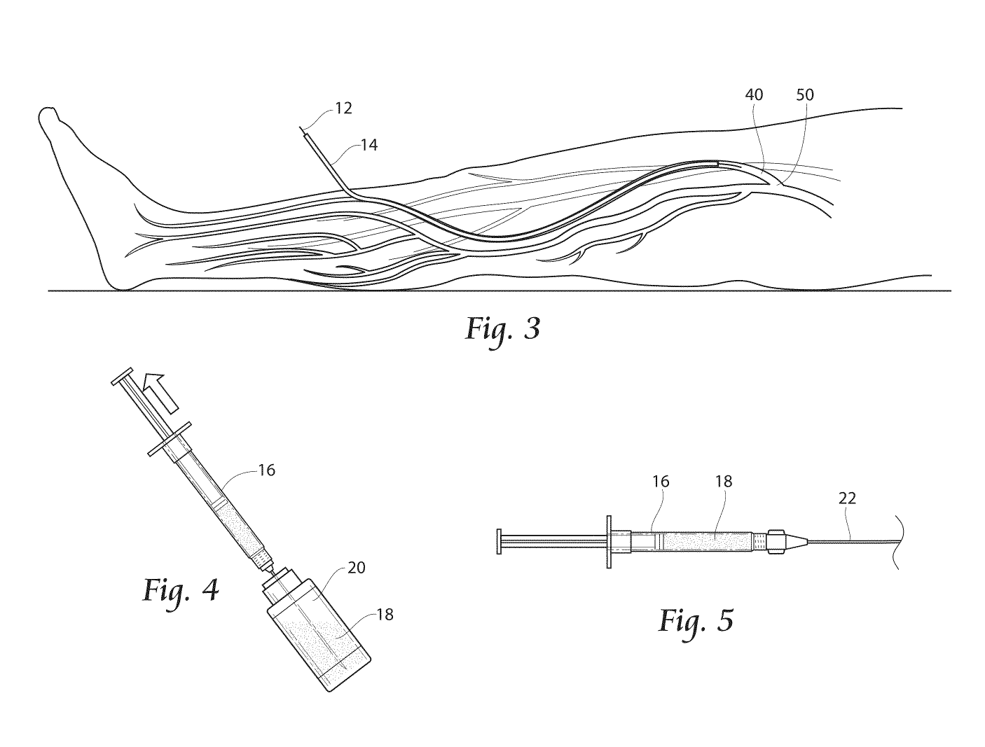

The guide wire 12 will be advance within the GSV 40 to an appropriate length. Once sufficiently positioned, the introducer sheath 14 is the inserted into the GSV 40 over the guide wire 12, as shown in FIG. 3. The introducer sheath 14 will allow for the catheter 22 to be inserted into the GSV 40. Once the introducer sheath 14 has been advanced a sufficient distance into the GSV, the guide wire 12 may then be removed.

As discussed above, the methods of the present invention are directed towards delivering the composition 18 that includes a photo-initiator material. FIG. 4 demonstrates the mixture 18 being loaded into the syringe 16 from the vial 20. It is understood that the syringe 16 could be any sufficient delivery device for the composition 18. An example of a syringe would be a 10 cc soft touch syringe. The vial 20 could be any sized container for storing the composition 18, but is typically a vial that will be a single use vial, for sterility purposes. For example, the vial 20 may be a 5 cc vial.

The composition 18 that will be delivered preferably comprises a material that includes a photo-initiator material in combination with an acrylate material. Preferably, the photo-initiator in the mixture 18 is an FDA approved photo-initiator that is highly efficient and sensitive to light at wavelengths provided by the light source 26. Not to he construed as limiting, examples of a photo-initiator preferred according to the present invention are Ciba® IRGACURE® 651, Alpha Hydroxyketone, and Ciba® Irgacure 819. The acrylate material is preferably an adhesive that exhibits bacteriostatic properties like a cyanoacrylate material, preferably either N-butyl Cyanoacrylate and 2-Octyl Cyanoacrylate or a combination thereof.

Referring now to FIG. 5, the catheter 22 is preloaded with the mixture 18 to preferably approximately 1 cm from the insertion end 24 of the catheter 22. Preloading the catheter 22 prior to insertion into the GSV 40 will much more efficiently insure that the mixture is properly delivered to a treatment area. However, it is understood that the syringe 16 could be used to deliver the mixture 18 into the catheter 22 after the catheter 22 is inserted into the GSV 40.

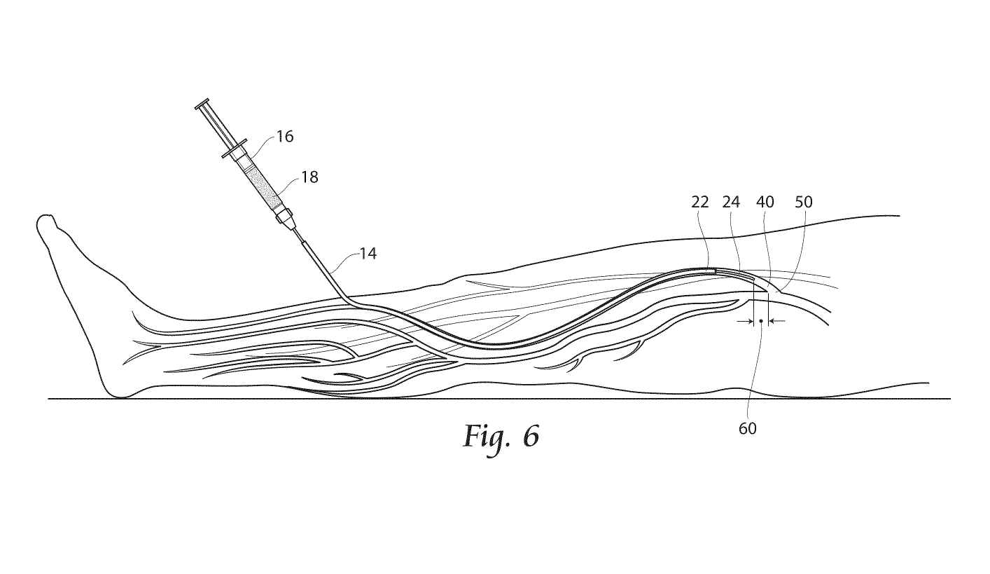

Referring now to FIG. 6, the catheter 22 is shown being advanced into the GSV 40. The catheter 22 is advanced so that the catheter insertion end 24 is placed in the introducer sheath 14, advanced to or near the patient's Saphenal-Femoral Junction (SFJ) 50, and located a first distance 60 distal, preferably approximately 3 cm, from the SFJ 50. Locating the catheter 22 the first distance 60 from the SFJ 50 reduces the likelihood that the mixture 18 will be introduced into the Femoral vein yet still provide adequate occlustion of the GSV 40.

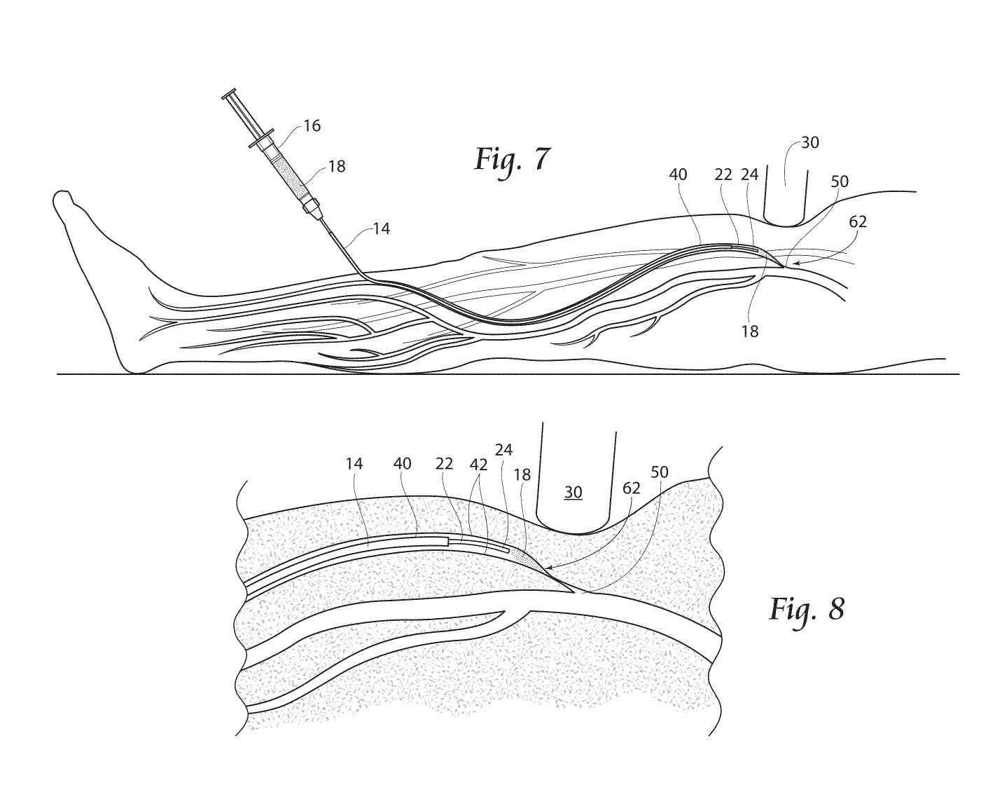

FIG. 7 depicts a first treatment area or a first occlusion portion 62 being established in the GSV 40. The first treatment area or a first occlusion portion is located between at or near the SFJ 50 and the catheter insertion end 24. The placing of the catheter insertion end 24 the first distance 60 distally from the SFJ 50 is preferably achieved by using ultrasonic guidance from an ultrasound transducer 30. However, any method of accurate placement now known or later discovered is included.

FIG. 8 depicts a close-up of the area 62. Once the area 62 is sufficiently confirmed, a pressure is applied to the GSV 40 and a first bolus of the mixture 18, preferably approximately 0.1 cc, is introduced into the GSV 40 at or near the first occlusion portion 62. The pressure is maintained or a first duration of time, preferably approximately 3 minutes.

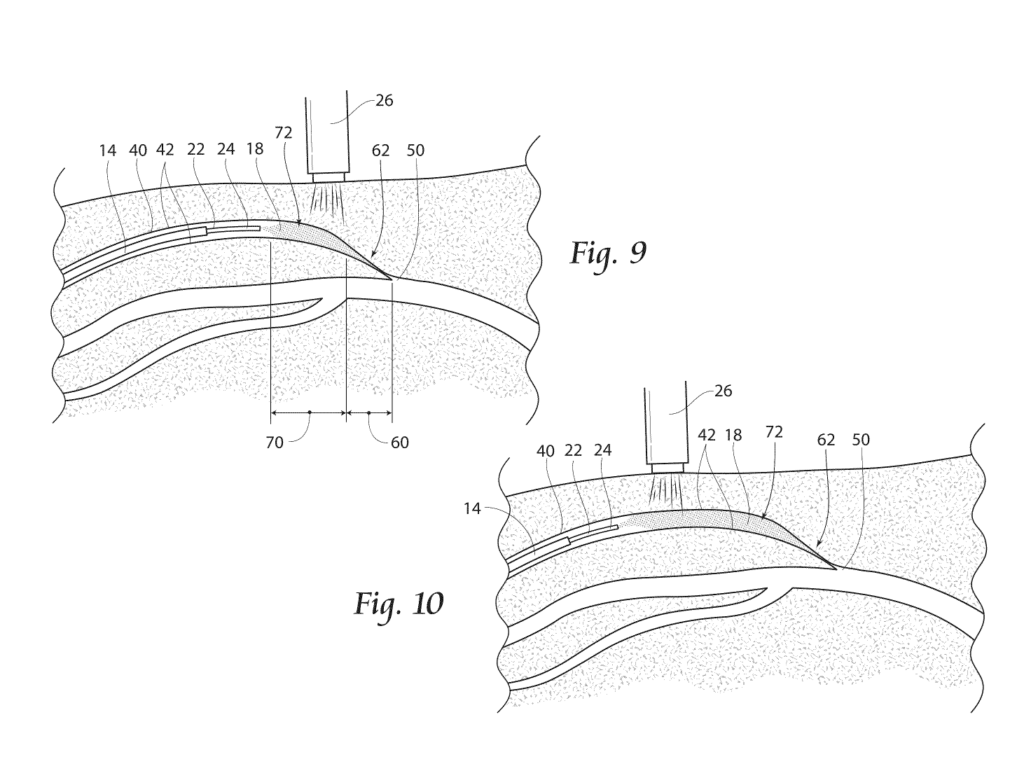

As depicted in FIG. 9, once the first bolus of mixture 18 has been cured or occluded, the catheter 22 and the catheter insertion 24 will be repositioned to a second treatment area or a second occlusion portion 72. The catheter 22 will be slowly withdrawn or retracted until the second treatment area or second occlusion portion 72 is reached. The second occlusion portion is established by introducing a second bolus of the mixture 18, preferably approximately 0.1 cc, in the GSV 40 over a second distance 70, preferably about 3 cm, at a slow and deliberate pace, and applying light from the LED light source 26 for a second duration of time, preferably approximately 30 seconds. The ultrasound transducer 30 may be repositioned to the location of the second occlusion portion 72 to ensure that the catheter 22 is properly located.

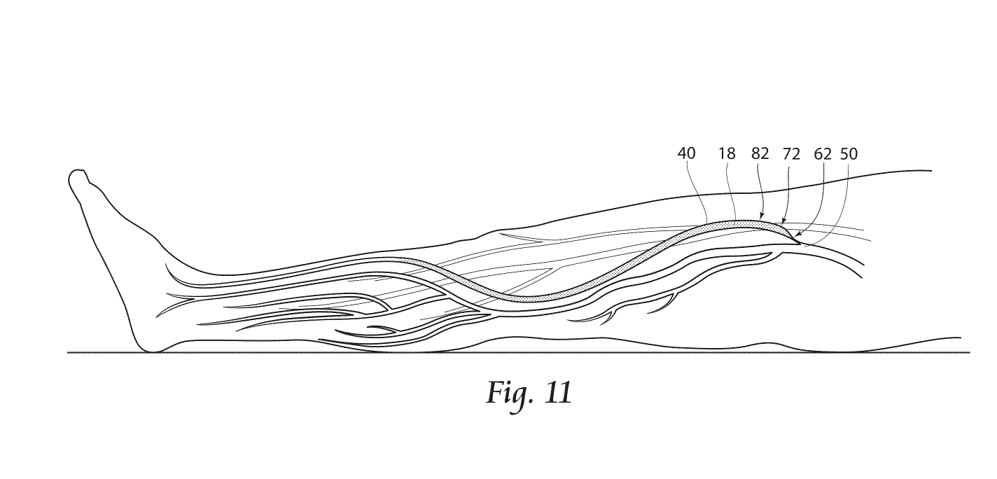

The step for establishing the second occlusion portion is repeated to form a plurality of treatment areas or occlusion portions 82 as required, as shown in FIGS. 10 and 11. The process will be repeated until the GSV 40 is sufficiently treated, as conditions indicate.

Once it has been determined that the GSV 40 is properly treated, the catheter 22 and the introducer sheath 14 may be removed. The resultant GSV 40 is shown in FIG. 11.

Previous techniques promoted creating bloodless intravascular environment in which the vein closure was achieved mostly by displacing the blood content and adhesively bonding the endothelial linings together. According to the present invention, the mixture 18 is preferably introduced into the GSV 40 without displacing the blood content (not shown) in the GSV 40. The photo-initiator in the mixture 18 cures through application of the LED light source 26 even when mixed with blood content present in the GSV 40.

The mixture 18 is preferably less viscous than previously used adhesives. Although low viscosity cyanoacrylate adhesives generally set up faster than medium and high viscosity cyanoacrylate adhesives, the mixture 18 is able to stay the curing of the mixture 18 because of the addition of the photo-initiator. The photo-initiator dilutes the cyanoacrylate in the mixture 18 and then promotes the curing of the mixture 18 when light from the LED light source 26 is applied.

The low viscosity provides for easier introduction of the mixture 18 and a more thorough coverage of, and adherence to, the endothelial linings 42 of the GSV 40, reducing treatment time and increasing bonding quality with the endothelial linings 42.

The procedure according to the present invention does not provide applying pressure at any occlusion site other than the first occlusion site 62. The mixture 18 is introduced in the GSV 40 with the existing blood (not shown) and cured in stages. Through the method according to the present invention, there is less potential for failure of the bonding formed between the endothelial linings 42 because the vein is more thoroughly filled and there are fewer coaptation points in the GSV 40 which could separate.

It is contemplated above that the ultrasound. transducer 30 applies the pressure to form the first occlusion portion 62, whereby the second occlusion portion 72 and the remaining occlusion portions 82 are formed by the application of light by the LED light source 26. However, because of the preferred curing properties of the mixture 18, application of light from the LED light source 26 may additionally or alternatively be provided to form the first occlusion portion 62. As stated above, the light induced curing properties of the mixture 18 provide for a more consistent occlusion of the GSV 40 and therefore may reduce the risk of detachment of the bond between the endothelial linings 42 due to insufficient pressure application during the curing phase.

The purpose of the system and method as disclosed herein is to provide a mixture 18 with a faster cure speed, thus allowing the patient's procedure time to be shorter and the bond between the endothelial linings 42 of the GSV 40 to be stronger, thus reducing the risk profile of the procedure.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I/We claim:

1. A method for treating a vein, the method comprising the steps of:
inserting a catheter into a vein having a distal end;
advancing the catheter within the vein to a first distance from the vein distal end;
establishing a first occlusion portion in the vein on the distal side of the catheter with a first amount of a mixture delivered through the catheter to the vein within the first distance, the mixture comprising a photo initiator and at least one of a N-butal Cyanoacrylate and a 2-Octyl Cyanoacrylate;
introducing a second amount of the mixture into the vein over a second distance;
establishing a second occlusion portion; and
removing the catheter from the vein.

2. The method of claim 1, wherein the first distance is approximately 3 cm.

3. The method of claim 1, wherein the second distance is approximately 3 cm.

4. The method of claim 1, wherein the first amount is approximately 0.1 cc.

5. The method of claim 1, wherein the second amount is approximately 0.1 cc.

6. The method of claim 1, wherein the step of establishing a first occlusion portion further comprises applying pressure to the vein for a first duration of time.

7. The method of claim 6, wherein the first duration of time is approximately 3 minutes.

8. The method of claim 1, wherein the second occlusion portion is formed by application of light from a light source for a second duration of time.

9. The method of claim 8, wherein the light source is an LED light source emitting light with a wavelength of approximately 400-500 nm.

10. The method of claim 8, wherein the second duration of time is approximately 30 seconds.

11. A method for treating a vein, the method comprising the steps of:
inserting a catheter into a vein having a distal end;
advancing the catheter within the vein to a first distance from the vein distal end;
establishing a first occlusion portion in the vein on the distal side of the catheter with a first amount of a mixture delivered through the catheter to the proximal side of the first occlusion portion, the mixture comprising a photo initiator and at least one of a N-butal Cyanoacrylate and a 2-Octyl Cyanoacrylate, the mixture mixing with blood present in the vein;
introducing a second amount of the mixture into the vein over a second distance, the mixture mixing with blood present in the vein;
establishing a second occlusion portion; and
removing the catheter from the vein.

12. The method of claim 11, wherein the first distance is approximately 3 cm. The method of claim 11, wherein the second distance is approximately 3 cm.

14. The method of claim 11, wherein the first amount is approximately 0.1 cc.

15. The method of claim 11, wherein the second amount is approximately 0.1 cc.

16. The method of claim 11, wherein the first occlusion portion is formed by applying pressure to the vein for a first duration of time.

17. The method of claim 16, wherein the first duration of time is approximately 3 minutes.

18. The method of claim 11, wherein the second occlusion portion is formed by application of light for a second duration of time.

19. The method of claim 18, wherein the light is provided by an LED light source emitting light with a wavelength of approximately 400-500 nm.

20. The method of claim 18, wherein the second duration of time is approximately 30 seconds.
